(19) United States
(12) Patent Application Publication
(10) Pub. No.: US 2008/0183110 A1
(43) Pub. Date: Jul. 31, 2008

(54) ULTRASOUND SYSTEM AND METHOD FOR HAIR REMOVAL

(76) Inventors: Scott A. Davenport, Half Moon Bay, CA (US); Gregory J.R. Spooner, Kensington, CA (US)

Correspondence Address: STALLMAN & POLLOCK LLP, 353 SACRAMENTO STREET , SUITE 2200, SAN FRANCISCO, CA, 94111, US

(21) Appl. No.: 11/851,351

(22) Filed: Sep. 6, 2007

Related U.S. Application Data
(60) Provisional application No. 60/824,610, filed on Sep. 6, 2006.

(51) Int. Cl. A61N 7/02

(52) U.S. Cl. 601/3

(57) ABSTRACT

In a method for removing hair using ultrasound, one or more pulses of ultrasound energy are applied to the tissue at a frequency (e.g. 5-15 MHz) selected to deliver the energy a tissue depths corresponding to those at which hair follicles are located. Pulse widths are selected to correspond to thermal relaxation times for hair. The skin may be optionally cooled before, during, and/or after ultrasound exposure.

This application claims the benefit of U.S. Provisional Application No. 60/824,610, filed Sep. 6, 2006.

### TECHNICAL FIELD OF THE INVENTION

The present invention relates to a system and method for removing hair using ultrasound energy.

### BACKGROUND

FIG. 1 illustrates, in simplified form, a hair 2 including a shaft 4 extending above skin surface 6 and a root 8 extending below the skin surface. The root 8 passes through epidermis 10 into dermis 12 with the base of the root being about 4 mm below surface 6. Root 8 is housed within hair follicle 14, hair follicle 14 being surrounded by various tissues including connective tissue sheath 16 and blood vessels 18. The various tissues closely surrounding root 8 and connected with the growth of hair 2, including hair follicle 14 and connective tissue sheath 16, are collectively referred to as hair tissue 20 in this application. The tissue of the pilosebaceous unit consists of dermal and epidermal tissues containing various amounts of vascular, connective, nerve and other tissue types, which contain collage fibers, melanin and hemoglobin, among other biomolecules. The hair shaft itself is composed largely of a protein called keratin. Hair color is primarily due to the presence of melanin in the hair. Melanin is created at the base of the hair follicle and is passed into the hair as it grows.

Various products exist that are designed for permanent or long-lasting removal of unwanted hair. The presence of melanin has made it possible to use lasers and other light sources for hair removal using wavelength-selective photo-thermolysis (SPTL) with melanin as the target chromophore. Using that process, the hair follicle and surrounding structure (referred to collectively as hair tissue) are selectively heated when the melanin in the hair tissue and in the hair root itself and is exposed to treatment radiation. The hair tissue is thermally damaged so that a result of the localized heating, many of the exposed hair units subsequently atrophy and are resorbed, sloughed from the epidermis, or remain present but disabled. While photo-thermolysis for hair removal relies upon limiting the flow of thermal energy to surrounding tissue during the applied radiation pulse by matching the thermal relaxation time, practical devices may include cooling of the skin surface before and/or during the treatment to minimize overall tissue damage or discomfort by active cooling of the skin.

Trancutaneous ultrasound approaches are presently used in medicine. Examples include 1-3 MHz collimated ultrasound fields for physiotherapy applications and non-invasive soft-tissue tumor therapy with high intensity focused ultrasound (HIFU).

The disclosed systems and methods use ultrasound energy for hair removal and are suitable as alternatives to selective photo thermolysis. Two approaches are described, one employing what is believed to be a selective acoustic mechanism that produces localized heating in the region of the hair tissue, and a second variant combining spatially selective and acoustically selective mechanisms.

The ultrasound absorption coefficient of a tissue is the percentage of incident ultrasound energy that is absorbed by the tissue (and converted to heat) instead of reflected or scattered by the tissue. The ultrasound absorption coefficient is a function of the mechanical properties of the tissue/biomolecules, the degree of scattering centers present (such as microbubbles), and the wavelength of the applied acoustic field. The actual absorption character of hair shafts and keratin biomolecules is not available in the scientific literature, as hair generally represents an obstacle to ultrasound imaging or therapy, rather than something to be targeted. It is known that the acoustic impedance of hair and keratin is greatly different than the surrounding soft tissues, which results in strong scattering/reflection of the applied acoustic field. It is also known that absorption of ultrasound in tissue greatly increases with increasing collagen content. And further, it is known that hair shafts and keratin contain very little collagen, and might thereby be expected to absorb little ultrasound energy directly. It would be expected that the heating rate of tissue surrounding hair would be only modestly greater than in the bulk tissue (or background rate) since the large acoustic impedance mismatch at the interface between the hair and surrounding tissue enhances the local acoustic field near heating rate at the surface of the hair. Surprisingly, the present inventors have found that continuous wave or pulsed ultrasound exposure to hairs in or on model tissue produces moderate-to-high heating of hair follicles rates under certain conditions, while not excessively heating the surrounding (i.e. to the point of damage). The inventors believe that these high heating rates are attributable to a localization of the acoustic energy as a result of some selective absorption of the ultrasound by the keratin in the hair, as well as the effects of reflection and scattering described above.

Use of ultrasound for hair removal provides an energy-based hair removal treatment that can be used for hair that is not sufficiently pigmented for SPTL treatment, such grey or white hair.

The onset of significant thermal damage to a hair follicle depends on the integrated temperature and time history that the follicle experiences. Typically, scientific or medical literature suggests that temperatures of 65 C or higher are required to begin damaging hair. This is typically true for exposures in which the heating pulse(s) match the thermal relaxation time associated with the hair structures. Lower temperatures can also produce damage to hair follicles if the exposures are much longer of the number of pulses is large.

### BRIEF DESCRIPTION OF THE DRAWINGS

FIG. 1 schematically illustrates a cross-section view of human skin, including a hair follicle, and further illustrates the focusing of an ultrasonic field in the region of the hair follicle in accordance with the disclosed methods;

FIG. 2 schematically illustrates an ultrasound hair removal system for use in practicing the disclosed methods;

FIG. 3 is an exploded view of one embodiment of an ultrasound applicator for the system of FIG. 2;

FIG. 4 is a cross-sectional side view of the ultrasound applicator of FIG. 3;

FIG. 5 is a block diagram schematically representing the system of FIG. 1;

FIG. 6A is a block diagram illustrating a first ultrasound driver system;

FIG. 6B illustrates RF output, RF drive, and bias gate waveforms using the driver system of FIG. 6A;

FIG. 7A is a block diagram illustrating a second ultrasound driver system;

FIG. 7B illustrates RF output, and bias gate waveforms using the driver system of FIG. 7A;

FIG. 8 is a cross-sectional side elevation view of an alternate ultrasound applicator for use in the system of FIG. 2;

FIG. 9 is a bottom plan view of the ultrasound applicator of FIG. 8.

FIG. 10A is a side elevation view of a second embodiment of an ultrasound applicator.

FIG. 10B is similar to FIG. 10A and shows a modification to the FIG. 10A transducer.

FIG. 11 is a side elevation view of a fourth embodiment of an ultrasound applicator.

FIG. 12 is a side elevation view of a fifth embodiment of an ultrasound applicator.

FIG. 13 is a cross-sectional side view of a modification to the first embodiment to incorporate the use of suction.

### DETAILED DESCRIPTION

FIG. 2 illustrates the general features of an ultrasound hair removal system 22. System 22 includes an ultrasound applicator 24 and a console 26 that includes an ultrasound power supply 28, a cooling system 30, and a controller 32. A handle 34 allows the applicator 24 to be glided over the body area to be treated.

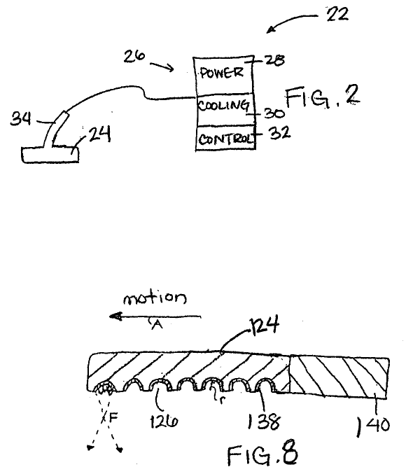

In a first embodiment, the system 22 uses ultrasound energy to induce selective and/or localized heating of hair sufficient to cause hair removal. FIGS. 3 and 4 show an ultrasound applicator 24a which may be used in this manner and that is suitable for use with the system of FIG. 2. Applicator 24a includes an ultrasound transducer 36 which comprises a tissue contact plate 38 and a piezoelectric crystal 39 (FIG. 4) formed of a piezoelectric material operable in a frequency range of approximately 1-30 MHz. Suitable piezoelectric materials include, but are not limited to, piezoelectric crystals known in the art as PZT, PT, K81, PNA, PNB, and HT. Crystal 39 has a flat shape that will produce a collimated energy pattern and that allows treatment of a large area at one time. In other embodiments, transducers shaped to produce divergent or convergent energy patterns might instead be used. Moreover, while a single transducer is shown, an array of transducers could be used and energized simultaneously or according to a desired pulse pattern (e.g. sequential pulsing).

The transducer 36 includes a substantially flat patient contact plate 38. An exemplary transducer has a contact surface that is circular and that has a diameter of approximately 2 cm. Contact plate 38 functions as an acoustic matching layer for the piezoelectric crystal 39 as well as a tissue cooling surface cooled by the system as discussed below. Tissue contact plate is formed of a material suitable for ultrasound transmission with sufficient thermal conductivity to allow superficial contact cooling of the skin. In one embodiment, tissue contact plate 28 is formed of aluminum having a gold coating on its tissue contacting surface. Other suitable materials for contact plate include, but are not limited to, bare aluminum, anodized aluminum, other metals such as copper, or thermally conductive crystalline solids such as sapphire or silicon nitride or boron nitride.

A printed circuit board 40 is electrically coupled to the transducer crystal via pin 43 and is connected to the console 26 (FIG. 2) by way of cable 42, thus providing the electrical interconnect between the crystal 39 and the cable 42.

Applicator 24a includes an applicator body 44 supporting the applicator components and providing a handle for the user to grasp during use. Printed circuit board 40 is seated within a recess 46 in the body 44. A retaining ring 48 holds the transducer 36 against the body 44 as shown in FIG. 4. Retaining ring 48 is attachable to the body 44 by screw threads that engage with corresponding threads on the distal portion of the body 44.

The handpiece may include cooling features for (1) cooling the surface of the skin while the underlying tissue layers are heated by ultrasound energy; and/or (2) removing heat generated in the handpiece during operation. In the illustrated embodiment, a thermoelectric cooler (TEC) 50 is mounted in contact with the body 44. The body 44, retaining ring 48 and contact plate 38 are formed of a thermally conductive material such as copper or aluminum, or others listed above, such that the TEC cooler 50 cools these structures, allowing for cooling of the handpiece and the tissue in contact with the contact plate 38.

A heat sink 52 positioned in contact with the back side of the thermo-electric cooler 50 draws away heat generated by the cooler 50. Heat sink 52 preferably includes micro-channels 54 through which cooling fluid circulates during use. The system may use feedback from sensors (not shown) in the handpiece to monitor the temperature of the ultrasound transducer and control operation of the cooling features to ensure adequate cooling. Because bone tissue can be heated very rapidly by ultrasound energy, some embodiments might include features that notify the user when the handpiece is positioned less than a predetermined distance from an underlying bone. For example, such a system might employ Doppler ultrasound to generate feedback corresponding to whether the handpiece is positioned within a certain distance from a patient's bone. For example, the system might detect the reflected ultrasound of the treatment pulse using a suitable transducer, or it might detect reflected ultrasound directed into the tissue using additional low power ultrasound transducers employed specifically for sensing the present of bone. These “diagnostic” transducers could operate at frequencies different from the treatment frequency to optimize resolution and/or allow filtering out of the reflected treatment ultrasound to increase the signal of the diagnostic ultrasound signal. In either case, the system analyzes the reflected ultrasound to generate feedback corresponding to whether the handpiece is positioned within a certain distance from a patient's bone. A time of flight type measurement might be made from a short duration or sharply switched ultrasound waveform. Alternatively, a simple amplitude or intensity measurement may suffice.

In such embodiments, feedback that the handpiece is near an underlying bone can result in a variety of responses. These responses include but are not limited to: (a) reducing the ultrasound intensity or terminating ultrasound delivery; (b) altering the ultrasound frequency (e.g. increasing the frequency so that that the energy is localized to shallower tissue regions); (c) causing an auditory and/or visual alarm; (d) and/or locking out the system against application of ultrasound until the handpiece is repositioned and/or the lock is overridden by the user. The transducer 36 may be used for bone sensing purposes, or the handpiece can include an additional transducer adapted specifically for bone sensing.

Additional sensors may be used to evaluate the sufficiency of ultrasound coupling between the contact plate and the skin using methods known in the art, such as for use in connection with ultrasound physiotherapy devices. For example, the system can measure the electrical impedance of the transducer amplifier. The measured impedance will increase if the transducer plate is not in contact with skin, for example. Other examples might instead be used, including include infrared proximity sensors. Feedback representing tissue contact may be used to reduce the ultrasound power to prevent overheating of the transducer.

An operational frequency for the transducer 36 is chosen to primarily limit ultrasound energy penetration to the tissues within which hair follicles are located. Thus, in a preferred mode of operation the transducer 36 is operable to create a heated zone of tissue that is sufficiently shallow to effect heating approximately 2 mm below the skin surface. Frequencies in the range of 5-15 MHz, and particularly those in the range of 5-10 MHz, have been found to be preferable for this purpose.

In general, increasing the ultrasound frequency will give shallower penetration, but the depth of penetration is further influenced by the amount of heat drawn from the skin using the cooling system, and the amount of ultrasound power used. The ultrasound peak power level is selected to be one that allows for heating of the hair tissue/follicle to a temperature that will cause damage to the hair sufficient for hair removal (believed to be approximately 65 C). Ultrasound peak powers in the range of 100-600 W/cm̂2 have been found suitable for this purpose.

If cooling is used, a cooling capacity is selected that keeps up with the evolution of heat to the surface, so that watts per square centimeter are “removed” at a particular temperature at which the skin surface is to be held. The combined effect of these parameters will give a thermal profile that is centered approximately 2 mm below the skin surface.

In order to produce a high localized temperature in the hair follicles, the transducer will ideally delivery acoustic energy within a relatively short time period. The ultrasonic pulse duration is selected to be comparable to the thermal relaxation time of the hair follicles, and ideally shorter than the thermal relaxation time of the surrounding tissue. Pulse durations in the range of 5-200 msec, preferably 10-100 msec, and more preferably in the range of 15-30 msec, have been found to produce optimal results. Generally thermal relaxation times for fine hair are shorter than those for more coarse hair. The system may allow the user to select pulse widths most suitable for the type of hair to be removed.

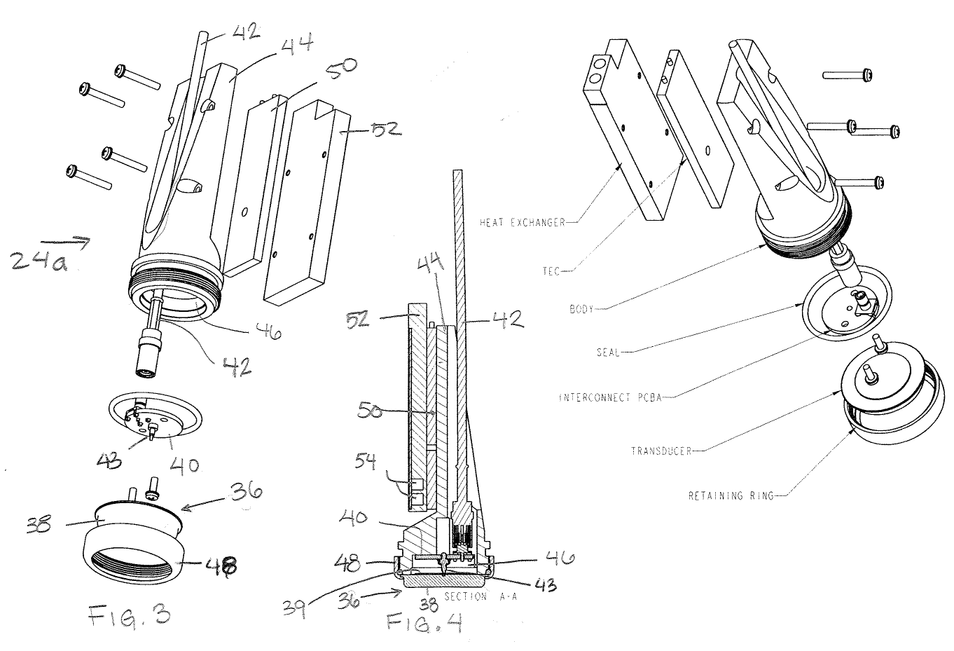

The system architecture for the system is illustrated in FIG. 5. The system includes the following main blocks: main processor board 54, main control board 56, LCD screen 58a, touch screen 58b, ultrasound generator board 60, hand piece 24, cooling system 64 and footswitch 65.

Main processor board 54 contains a main microprocessor 55 having an associated memory and input/output ports. Microprocessor 55 controls graphical user interface (GUI) features drawn on the system's LCD screen 58a, receives user input (e.g. treatment parameters) from the touch screen 58b and communicates with the main control board 56 and an electrically isolated hand piece processor 66. The main microprocessor 55 and the main control board 56 communicate via a bidirectional serial link 68. Another bidirectional serial link 70 transmits communications between the hand piece processor 66 and the main microprocessor 55.

The main control board 56 governs most of the system's hardware functionality. Main control board 56 includes a main control CPU 72, safety control CPU 74 and all necessary input/output ports. The main control CPU 72 receives commands from the main microprocessor 55 via serial link 68. Commands include exposure settings and limits, status requests and auxiliary commands.

Main control CPU 72 also maintains communication with safety control CPU 74 via a bidirectional serial link 76. Both of the control CPUs 72, 74 monitor the system footswitch 65 which is engaged by a user to activate treatment.

Main control CPU 72 controls the ultrasound generators 80 on the ultrasound generator board 60, and monitors the ultrasound power signal generated on the ultrasound generator board 60.

The safety control CPU 74, among other system tasks, monitors the ultrasound power signal generated on the ultrasound generator board 60, thus implementing a redundant power monitoring system.

The hand piece processor 66 receives commands from the main microprocessor 55 and executes temperature control tasks. This system controls the TEC (thermoelectric cooler) 50 located in the hand piece 24 based on temperature feedback signals needed for closed loop control. Handpiece processor 60 may also receive feedback corresponding to bone detection and/or contact sensors. That feedback is used by the main CPU 72 to modify ultrasound parameters as needed.

Ultrasound generators and amplifiers 80 provide drive signals for the ultrasound transducer 36. In a preferred embodiment, an RF driver is used to generate drive signals in the RF frequency range. When the drive signals are provided to the transducer crystal, the transducer emits acoustic energy from its exposed surface, as is well known to those skilled in the art. The system may include a driver for the ultrasound transducer that will operate the transducer at its fundamental frequency and/or at one or more of its overtones or harmonics.

In order to practically provide the high peak RF power levels expected (3-9 kW) at short duty factors (1-5%) with reasonable overall efficiency, the RF drive source must use higher voltage switching elements, and a gated bias scheme. Some degree of frequency agility will also be required to compensate for manufacturing tolerances in the ultrasound transducers, perhaps as much as 30%. A Class-B amplifier or a Class-C push-pull amplifier design will accommodate this frequency agility while maintaining a reasonable efficiency. If tighter transducer manufacturing tolerances are available (to about 5%), a Class-D amplifier will be preferable.

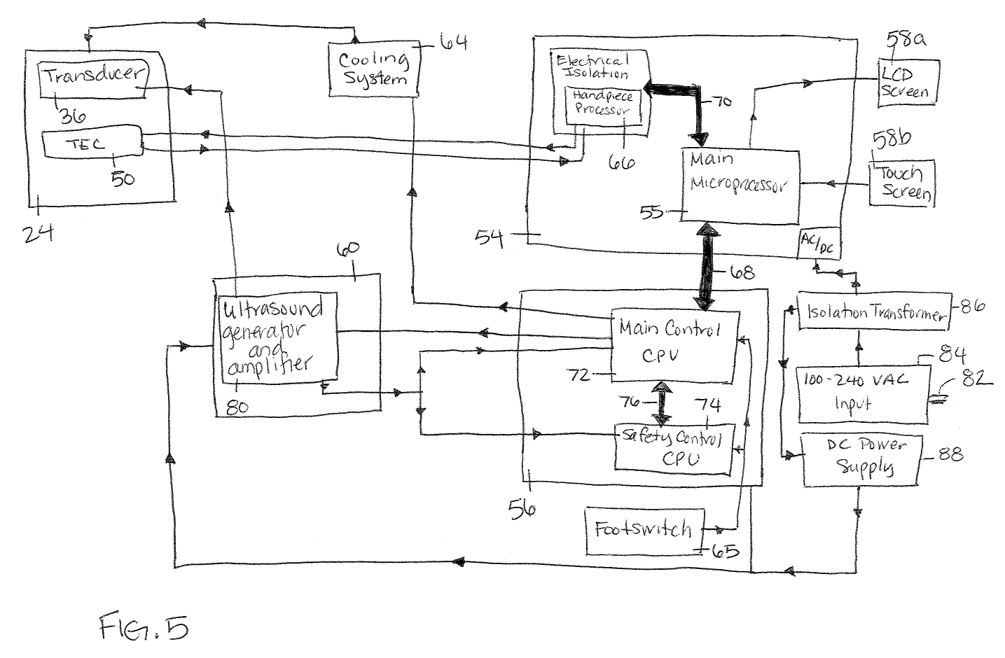

One design for an RF drive system using a Class-C amplifier uses a “master oscillator/power amplifier” (MOPA) type system illustrated in FIG. 6A. According to this embodiment, the RF driver includes a master oscillator 90, a linear preamplifier stage 92, a driver stage 94, and a final amplifier stage 96 (i.e. the Class-C amplifier). The preamplifier and drivers can use a constant bias supply 98, set for linear operation. The final amplifier 96 will be biased for Class-C operation, with an inter-pulse blanking feature that zeroes the large quiescent current in the final amplifier stage between pulses. This will reduce the total amplifier power dissipation by >90%, compared to a continuous wave amplifier with the same peak power capacity. The amplifier is configured to produce peak drive amplitudes of nearly 1000 volts. The output is passed through a reactance tuning circuit 100 that tunes out the transducer's capacitance at the natural frequency of the transducer.

In normal operation, an enable signal from a timing and control system 102 will turn on the bias supply to the final stage about 1 msec before the RF drive signal as shown in FIG. 6B, in order to allow the circuit to stabilize. Afterwards, the RF drive signal is applied for the desired pulse duration. The bias enable signal is removed immediately after the RF pulse terminates. This cycle is repeated at the desired repetition frequency, typically about 2 Hz.

Although the final amplifier elements are electrically DC in parallel, a multi-stage set of combiners will effectively add their RF output voltages in series. The output combiners can set the final output impedance from 25-75 ohms, depending on the optimal drive impedance for the acoustic transducer.

The control system 102 for the ultrasound driver will measure the output drive voltages and currents, and determine the optimum drive frequency fed to the RF amplifier stages. For example, the control system 102 will monitor the phases of the voltages and currents and sample various drive frequencies until the optimal frequency is found that will bring the monitored voltage and current into phase. In this way, the driver system is responsive to variations between the natural frequencies of different ultrasound transducer crystals, and to changes in the natural frequency of a transducer that can occur during use (e.g. as a result of load variations and/or heating of the crystal).

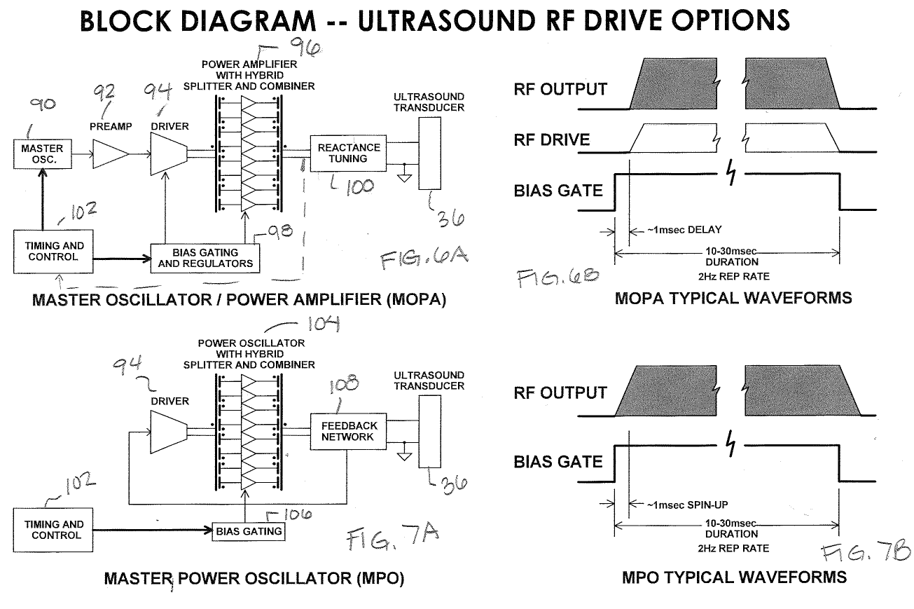

An alternate RF drive system shown in FIG. 7A incorporates a master power oscillator design. This system includes a driver 94 that applies drive signals to a power oscillator 104. A bias gating circuit 106 applies an enable signal to the power oscillator 104. This system used feedback from feedback system 108 to cause the oscillator 104 to oscillate at the frequency at which the highest gain is produced, which corresponds to that at which the transducer is most resonant. The RF output reaches its maximum after a spin-up time of approximately 1 msec as shown in FIG. 7B.

As discussed previously, the cooling system 64 includes a heat exchanger 52 (within the handpiece as shown in FIG. 4), together with a water reservoir and a pump. This system is designed to remove heat created in the hand piece during operation as well as enable skin temperature control facilitated by the TEC 50. It is controlled by main control CPU 72

System AC input comes from an AC wall plug 82 to input module 84.

Isolation transformer 86 feeds both the DC power supply 88 and on-board DC power supply located in the main processor board 54.

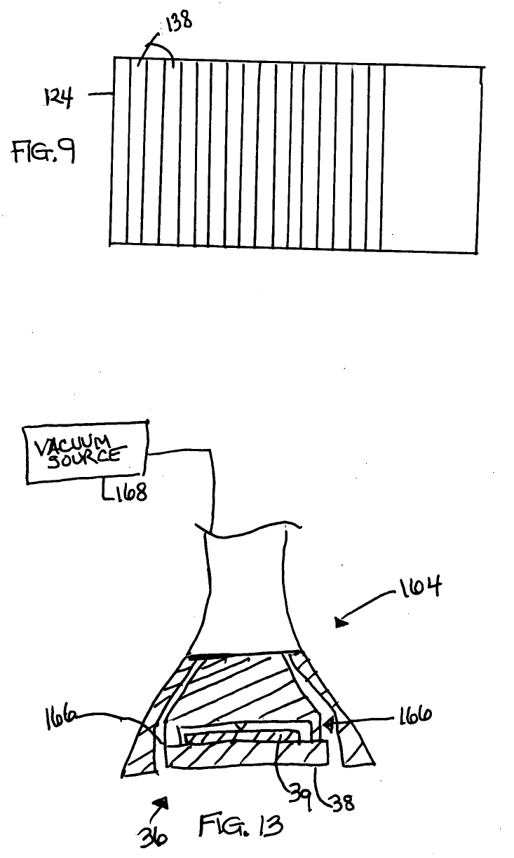

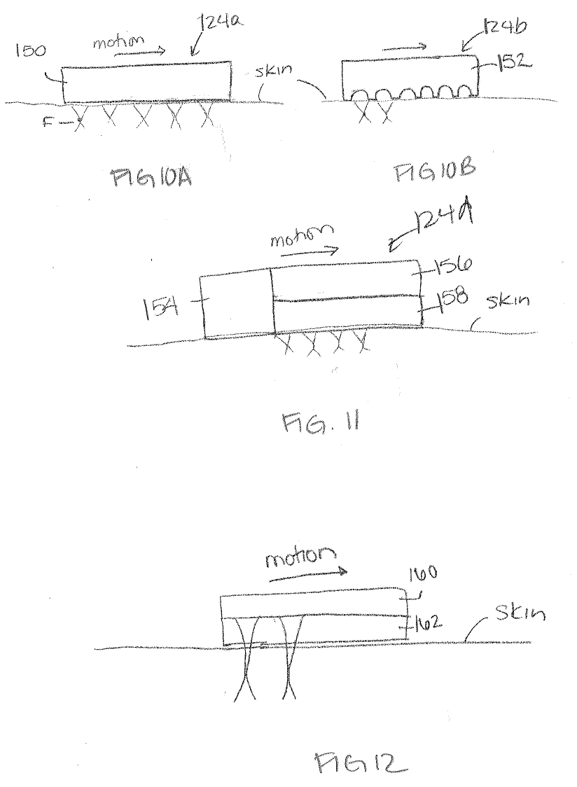

In a modification to the FIG. 3 embodiment, the ultrasound applicator may be modified to apply suction to the hairs undergoing ultrasound treatment, so as to locally increase the concentration of ultrasound energy in the hair follicles. In such an embodiment, the use of vacuum pressure ensures that the deeply rooted hairs are raised during ultrasound application, allowing the bulb of the hair to elevate to a depth that will be targeted by the penetrating ultrasound at higher frequencies. In one example schematically illustrated in FIG. 13, handpiece includes a fixation cup 164 positionable in contact with a patient's skin over the area to be treated. The ultrasound transducer 36 is positioned within the fixation cup. Vacuum ports 166 within the cup are coupled to a vacuum source (discussed in connection with FIG. 5), such that application of suction via the ports 166 will draw a patient's hair and/or skin into contact with the tissue contact plate 38 and to optionally temporarily fix the cup 164 against the skin. Cooling may be used as described above. Details concerning a vacuum system suitable for use with the handpiece 164 are shown and described in U.S. application Ser. No. 11/851,335, (Attorney Docket: ALTU-2310), entitled SYSTEM AND METHOD FOR DERMATOLOGICAL TREATMENT USING ULTRASOUND, filed Sep. 6, 2007 and incorporated herein by reference.

During use of the system of FIGS. 2-7B, an acoustic coupling material such as water or gel may be applied to the skin or the applicator to optimize acoustic coupling between the contact plate and skin. The contact plate 38 is placed in contact with the skin in the region to be treated. Footswitch 65 is depressed to activate ultrasound energy delivery.

In one mode of operation, a single pulse of ultrasound energy is delivered to the tissue at the appropriate frequency (e.g. 5-15 MHz), intensity (e.g. 100-600 W/cm̂2) and pulse width (e.g. 10-100 msec) as discussed above to heat hair follicles within the region to a temperature sufficient to damage the hair follicles such that the hairs will fall out and/or discontinue growth. In alternate embodiments, between 1 and 10, and more preferably between 1 and 5 pulses are delivered to the tissue region to effect the desired amount of heating of the hair follicle. The skin may be optionally cooled before, during, and/or after ultrasound exposure using the TEC 50 to cool the contact plate 38. Following ultrasound exposure, the contact plate 38 is repositioned on a different part of the skin and the process is repeated until an entire area over which hair removal is desired has been treated. This method may be modified for use in connection with the FIG. 13 embodiment to incorporate the step of applying suction through the ultrasound applicator to enhance energy delivery as disclosed.

Experiments using a system of the type shown in FIG. 3 demonstrate that high hair structure temperatures can be achieved in a single pulse, if the intensity is high (I>100 W/cm̂2). For integrating thermal damage in one or more pulses, hair damage may be produced with the following approximate schedule:

| d | n | I | f | t | D | ΔTn | Tc |
|---|---|---|---|---|---|---|---|
| 5 mm | 1 | 150 W/cm\hat{ }2 | 5 Mhz | 10 ms | N/A | 20 C. | 25 C. |
| 5 mm | 1 | 300 W/cm\hat{ }2 | 5 Mhz | 10 ms | N/A | 40 C. | 25 C. |
| 2.5 mm | 1 | 150 W/cm\hat{ }2 | 10 Mhz | 10 ms | N/A | 30 C. | 25 C. |
| 2.5 mm | 1 | 300 W/cm\hat{ }2 | 10 Mhz | 10 ms | N/A | 60 C. | 25 C. |
| 5 mm | 5 | 150 W/cm\hat{ }2 | 5 Mhz | 10 ms | 1% | 35 C. | 25 C. |
| 5 mm | 5 | 300 W/cm\hat{ }2 | 5 Mhz | 10 ms | 1% | 55 C. | 25 C. |
| 2.5 mm | 5 | 150 W/cm\hat{ }2 | 10 Mhz | 10 ms | 1% | 45 C. | 25 C. |
| 2.5 mm | 5 | 300 W/cm\hat{ }2 | 10 Mhz | 10 ms | 1% | 75 C. | 25 C. |

Where:
d = depth of the heating zone
I = ultrasound intensity
f = ultrasound frequency
t = pulse or burst duration
D = duty cycle
n = number of pulses
Tc = temperature of contact cooling surface
ΔTn = temperature rise of the hair follicle after ‘n’ pulses

FIGS. 8-12 illustrate features that may be used in an alternative ultrasound hair removal system. According to use of the alternative system, to assist in minimizing damage to the surrounding tissue, pulsed energy is delivered, with each pulse having a duration corresponding approximately to the thermal relaxation time for hair, or approximately 25-150 msec. In accordance with this embodiment, when pulsed ultrasound having a pulse width of approximately 50 msec is used, an acoustic intensity of approximately 100-600 W/cm² at the hair follicle is beneficial for elevating the temperature of the follicle by an amount sufficient for hair removal.

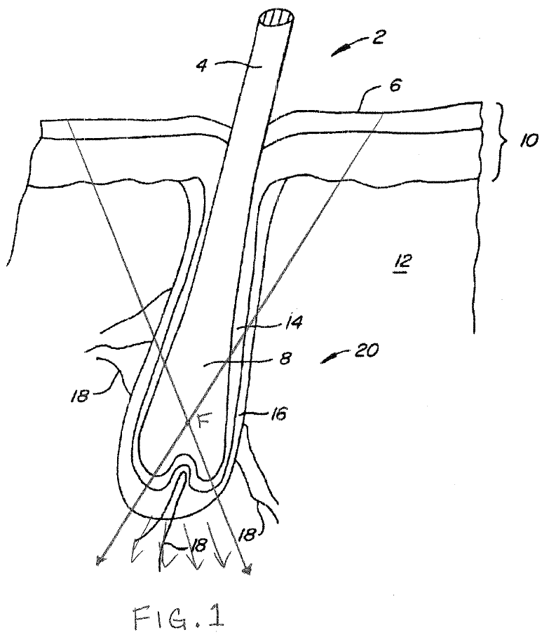

To achieve an intensity of this magnitude, the system 22 is designed to focus high intensity ultrasound in the region of the hair follicle as illustrated by the field lines in FIG. 1. FIGS. 8 and 9 illustrate one example of an ultrasound applicator 124 for delivering focused ultrasound. For simplicity, the conductors for supplying electrical current to the transducers of the applicator are not shown.

In an embodiment shown in FIG. 8, applicator 124 includes a plurality of semi-cylindrical depressions 136. Each depression 136 comprises a separate ultrasound transducer, and thus contains one or more piezoelectric elements 138 arranged to deliver ultrasound energy towards a focus F (which will be a line of focused energy running parallel to the depressions 136, given the semi-cylindrical geometry). Each depression 136 contains a single semi-cylindrical piezoelectric element 38 that lines the depression 136, however multiple elements of other shapes and sizes may instead be used. For example, piezoelectric elements may be longitudinally and/or circumferentially arranged within the depressions 136. In other embodiments, the semi-cylindrical depressions may be replaced with depressions that have other geometries that will similarly provide for focused ultrasound transmission, such as semi-spherical or parabolic geometries. In other embodiments, the applicator might instead use a phased array of transducer elements (see planar array 150 described in connection with FIG. 10A) in which focusing is provided for by controlling the phase of individual transducer elements. In alternate embodiments, external focusing elements (see lens array 158 described in connection with FIG. 11) can be used.

In the illustrated embodiment, the semi-cylindrical depressions are preferably formed with a radius r (FIG. 8) of approximately 1-4 mm, and most preferably approximately 3 mm. With this geometry, intensity levels of up to 600 W/cm² are achievable at the hair follicle while maintaining a surface intensity of approximately 3 W/cm² at a frequency range of approximately 1-5 MHz.

The applicator 124 preferably includes a cooling element 140 for cooling the skin before, during and/or after delivery of the ultrasound energy. For example, cooling element 140 may be a thermally conductive material such as copper or sapphire. Cooling system 130 may function to circulate a cooling fluid through cooling lumens (not shown) in the cooling element 140 and/or applicator 124, and/or a thermoelectric cooling device may be operated by the cooling system to cool the cooling element 140, transducers 138 and/or another portion of the applicator 124 that is to be placed in contact with the skin.

During use of the system 122, an acoustic coupling material such as water or gel may be applied to the skin or the applicator to optimize acoustic coupling between the transducers and skin. The transducers 138 are energized as the applicator 124 is moved across the skin in an area from which hair is to be removed, causing the transducers to emit focused ultrasound energy into the tissue. The cooling system may be activated prior to ultrasound delivery to pre-cool the skin, and/or it may be activated during ultrasound delivery. In the FIGS. 8 and 9 embodiments, the cooling system operates to cool the skin via cooling element 140. In this embodiment, cooling element 140 is positioned so that during movement of the applicator across a treatment area, the cooling element 140 moves over the target area, cooling the target area following ultrasound exposure of that area. In alternative embodiments, the cooling element 140 may be positioned to pass over the target tissue before ultrasound exposure, or cooling elements may be positioned for both pre-exposure and post-exposure cooling.

In alternative embodiments, the applicator may include a transducer array that forms both cooling and ultrasound transmission functions. For example, applicator 124a of FIG. 10A includes a transducer 15Q that is both a planar phased array as well as a cooling element cooled by cooling fluid channels or thermoelectric coolers. In the FIG. 10B embodiment of an applicator 124b, the phased transducer array of FIG. 10A is replaced by an array 152 of semi-cylindrical transducer channels that focus energy as discussed in connection with FIG. 8. In this embodiment, as with FIG. 10A, the transducer performs both cooling and ultrasound transmission functions, allowing simultaneous cooling and ultrasound transmission to underlying tissue.

Another embodiment shown in FIG. 11 is similar to the FIG. 8 embodiment in the sense that the cooling element 154 is positioned at the trailing (or leading, if desired) end of the applicator 124c, relative to the applicator's direction of motion. In this case, however, a planar transducer array 156 is positioned to direct ultrasound energy through a lens 158 that focuses ultrasound energy to tissue. Suitable lens materials include low velocity materials (relative to acoustic velocity in water) such as silicone rubbers including GE RTV 560, or higher velocity materials such as TPX.

In another variation shown in FIG. 12, the applicator 124d includes a transducer 160 positioned to transmit ultrasound through a cooling element 162 formed of material having good thermal conductivity (e.g. metals, such as copper or aluminum, or other crystalline solids such as sapphire). Here the transducer 160 may have focused transducer elements of the type shown in FIG. 8, or a phased array as in FIG. 10A. In these embodiments, the focusing of the ultrasound energy is adapted to accommodate any changes to the ultrasound wavefront that would be presented by the cooling element. In an alternative embodiment, the cooling element 162 may function as a lens to focus the energy as in FIG. 11.

In a further modification to the disclosed embodiments, the piezoelectrics may be driven to produce a focused beam of ultrasound energy that scans across the tissue, thus eliminating the need to move the applicator across the tissue surface. During use of this type of embodiment, the applicator would be placed against the skin and left in place while the ultrasound energy scans along the target area. The applicator would then be repositioned to an adjacent tissue area and the process repeated until treatment of the entire area is completed.

It should be recognized that a number of variations of the above-identified embodiments will be obvious to one of ordinary skill in the art in view of the foregoing description. It should be appreciated, moreover, that the various features of the embodiments that have been described may be combined in various ways to produce numerous additional embodiments. Accordingly, the invention is not to be limited by those specific embodiments and methods of the present invention shown and described herein. Rather, the scope of the invention is to be defined by the following claims and their equivalents.

Any and all patents, patent applications and printed publications referred to above, including for purposes of priority, are incorporated by reference.

1. A hair-removal method comprising:
delivering a pulse of ultrasound energy having a pulse duration of 5-200 msec through skin to cause thermal damage to an underlying hair follicle.

2. The hair removal method of claim 1, further including the step of positioning an ultrasound applicator in contact with the skin and delivering the pulse from the ultrasound applicator.

3. The method of claim 2, further including cooling the skin in contact with the ultrasound applicator to cause a reverse thermal gradient between the target tissue and the skin.

4. The method of claim 1, wherein the ultrasound is delivered using an ultrasound frequency selected to create heat in a tissue area containing hair follicle.

5. The method of claim 4, wherein the tissue area includes tissue at a depth of 1-6 mm from the skin surface.

6. The method of claim 5, wherein the depth is approximately 2-3 mm from the skin surface.

7. The method of claim 5, wherein the ultrasound causes heating to at least approximately 65 C at the depth.

8. The method of claim 4, wherein the ultrasound frequency is in a range of 5-15 MHz.

9. The method of claim 8, wherein the ultrasound frequency is in a range of 5-10 MHz.

10. The method of claim 1, wherein the pulse width is selected to approximately match a thermal relaxation time of hair.

11. The method of claim 1, where the ultrasound energy is delivered using a pulse having a pulse width of approximately 10-100 msec.

12. The hair-removal method of claim 1, wherein the applying step includes the step of moving the ultrasound applicator along the surface of the skin during energy delivery.

13. The hair-removal method of claim 1, wherein the method includes the step of applying suction to the skin during ultrasound delivery to draw a plurality of hair follicles in a direction towards the ultrasound applicator.

14. The hair-removal method of claim 1, wherein the method includes applying focused ultrasound energy.

15. The hair-removal method of claim 1, wherein the applying step applies an acoustic intensity in the range of 100-600 W/cm² to the hair follicle.

16. The hair-removal method of claim 1, wherein the applying step applies an acoustic intensity of 1-20 W/cm² to the skin overlaying the hair follicle.

17. The hair-removal method of claim 2, wherein the method includes the step of applying ultrasound energy through a cooling element to the skin of the patient.

18. The hair-removal method of claim 17, further including the step of cooling the skin using the cooling element while applying ultrasound energy through the cooling element.

19. The hair removal method of claim 2, wherein delivering ultrasound energy includes, with the ultrasound applicator at a first tissue location, delivering only a single pulse of ultrasound energy, said pulse resulting in damage to a plurality of hair units sufficient to cause atrophy of the damaged hair units.

20. The hair removal method of claim 19, wherein said single pulse elevates tissue in the region of the hair units to at least 65 C or higher.

21. The hair removal method of claim 19, further including repositioning the ultrasound application to a second tissue location following delivery of the single pulse and delivering a second pulse at the second tissue location.

22. The hair removal method of claim 2, wherein delivering ultrasound energy includes, with the ultrasound applicator at a first tissue location, delivering no more than 10 pulses of ultrasound energy, said pulses resulting in damage to a plurality of hair units sufficient to cause atrophy of the damaged hair units.

23. An ultrasound hair removal system comprising:
an ultrasound device including a power source and an ultrasound applicator comprising an ultrasound transducer and a skin contact plate positionable in contact with skin; and
a control system electronically coupled to the ultrasound device, the control system operable to cause the ultrasound device to deliver a pulse of ultrasound energy having a pulse width in the range of approximately 10-100 msec.

24. The system according to claim 23, wherein the pulse has a pulse width in the range of approximately 15-30 msec.

25. The system according to claim 23, wherein the transducer is operable to deliver ultrasound power having a frequency in the range of 5-15 MHz.

26. The system according to claim 23, further including a cooling element in contact with the skin contact plate.

27. The system according to claim 23, wherein the control system is operable to cause the ultrasound device to deliver ultrasound energy having intensity in the range of 100-600 W/cm̂2.

28. The system according to claim 23, wherein the ultrasound device has a planar sound transducer.

29. The system according to claim 23, wherein the ultrasound device has a focused ultrasound transducer.
